(19) United States
(12) Patent Application Publication
(10) Pub. No.: US 2017/0258369 A1
(43) Pub. Date: Sep. 14, 2017

(54) LARYNGEAL MECHANOSENSOR STIMULATOR

(71) Applicant: The University of Toledo, Toledo, OH (US)

(72) Inventors: Reginald Baugh, Ann Arbor, MI (US); Ali Hassani, Ann Arbor, MI (US); Payam Entezami, Toledo, OH (US)

(73) Assignee: The University of Toledo, Toledo, OH (US)

(21) Appl. No.: 15/590,102

(22) PCT Filed: Nov. 19, 2014
(86) PCT No.: PCT/US2015/061282

§ 371 Date: May 9, 2017

Related U.S. Application Data
(60) Provisional application No. 62/081,802, filed on Nov. 19, 2014.

(51) Int. Cl. A61B 5/11; A61B 5/00

(52) U.S. Cl. CPC A61B 5/1104; A61B 5/0053; A61B 5/0057; A61B 5/4005

(57) ABSTRACT

This invention is a device that provides feedback controlled pressure output that delivers a highly controlled and accurate air stimulus into the larynx for diagnostic use. The device is designed to be significantly more accurate, safer for the patient, and highly modular for easy future modifications compared to previously available commercial devices.

### CROSS REFERENCE TO RELATED APPLICATIONS

This application claims the benefit of U.S. provisional patent application Ser. No. 62/081,802 filed on Nov. 19, 2014.

### TECHNICAL FIELD OF THE INVENTION

The invention is a device for a feedback controlled pressure output system designed to deliver a highly controlled and accurate air stimulus into the larynx coupled with mucosal sensory monitoring for diagnostic use.

### BACKGROUND OF THE INVENTION

Proper larynx function is critical in our daily lives. Part of its job is to instinctively close the glottic airway and initiate a cough in response to a foreign body, such as food or liquid, entering the trachea. Should this instinctive response fail to elicit properly, aspiration of the foreign body into the lungs can occur, with a significant risk of subsequent infection called aspiration pneumonia. Aspiration pneumonia has a mortality rate as high as 70%, and accounts for 10 to 30 percent of all deaths associated with anesthesia. Risk of aspiration is increased in patients with a wide variety of pathologies from gastric acid reflux to neurological diseases and stroke. Specifically, patients undergoing surgical operations under general anesthesia and intubation have been shown to have a deceased ability to achieve full laryngeal reflex post-operatively for an unknown amount of time.

While the laryngeal reflex eventually returns to normal, in an effort to avoid aspiration complications, surgery departments across the world have placed protocols dictating when a patient is allowed to resume eating and drinking after surgery. However, this time period has so far been arbitrarily set by each department, with little insight as to when the laryngeal reflex actually returns.

Thus the inventors came up with the idea to research the return of the laryngeal reflex post-operatively, by creating a novel air stimulus device for experimental and diagnostic purposes.

### SUMMARY OF THE INVENTION

This invention is an extremely precise air stimulus that uses a control system to output medical grade air into the larynx, while simultaneously monitoring mucosal sensory activity for diagnostic use. The presentation of air stimuli has been widely used in the past in order to better understand the actions of the larynx which include swallowing, speech, and protection. However, no prior air stimulus device exists on the market currently. There previously existed a popular commercial device, the Pentax AP-4000, but that has been out of production (and service) for a number of several years. It is now recognized as inaccurate and unsuitable for research. As such, it fell out of favor due to its limited clinical value. Now, the door is open for the device of this invention to go well beyond filling its place. Our device is designed to be significantly more accurate, safer for the patient, highly modular for easy future modifications and provide diagnostic information not previously available.

This invention delivers a novel tool that is very precise, safe and modular permitting easy future improvements. The first requirement is that it must be a feedback controlled device. Feedback control provides output stability, faster ramp times (the time it takes to reach 2% error from the desired output) and more robustness (the ability to be compatible with all types of patients.) The ability to simultaneously monitor pharyngeal sensory function shall provide clinics with unmatched diagnostic accuracy. The second requirement is safety. This is accomplished by building a closed and carefully monitored system, using a medical air source as our pressure source and components that could be easily sterilized. In this manner, no contamination would be introduced into the patient. In addition, there are both hard coded delimiters and mechanical relief valves that prevent unsafe pressures from being administered. This combination of sterility and safety mechanisms shall both actively prevents users from unintentionally harming their patients and reduces any risks of infection. Lastly, the third requirement is that the device be modular, such that additional inputs and outputs could be programmed in the future. The selected microcontroller is a float point grid array capable of many task. This provides the ability to write code that is very flexible, such that we not only have our primary control function, but can easily take advantage of new signals. An example of how we have taken advantage of this is by monitoring the pharyngeal mucosa sensory function. In addition, we have created a digital Guided User Interface, rather than providing the user with an analogue set of haptic inputs (i.e. dials and buttons). By creating a software human-machine-interface, we can continuously upgrade the backend software to add more functionality, and respond to customer feedback to make the device more user-friendly. We believe this will not only provide greater functionality, but also lead to a more satisfactory customer experience.

In one embodiment, an ultrasonic sensor is integrated into the laryngeal mechanoreceptors.

Other objects and advantages of the present invention will become apparent to those skilled in the art upon a review of the following detailed description of the preferred embodiments and the accompanying drawings.

### IN THE DRAWINGS

FIG. 1 is a view of a prior art apparatus.

FIG. 2 is a view of a laryngeal mechanosensor stimulator according to this invention.

FIG. 3 shows a sensory electrode for measuring laryngeal-nerve input.

FIG. 4 shows an ultrasonic sensor integrated into the laryngeal mechanoreceptors.

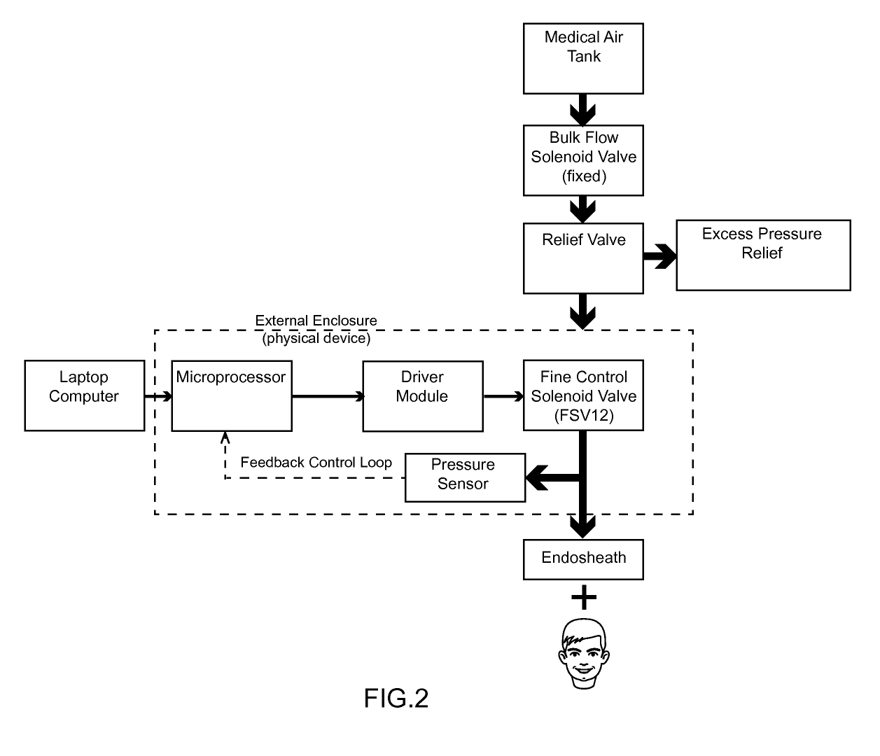

The device schematics shown in FIGS. 2-4 include the following components.

### LEGEND

Dashed line=external enclosure
Filled arrow=electrical connection
Hollow arrow=air connection
Dashed arrow=feedback line
+=patient connection

### DETAILED DESCRIPTION OF THE INVENTION

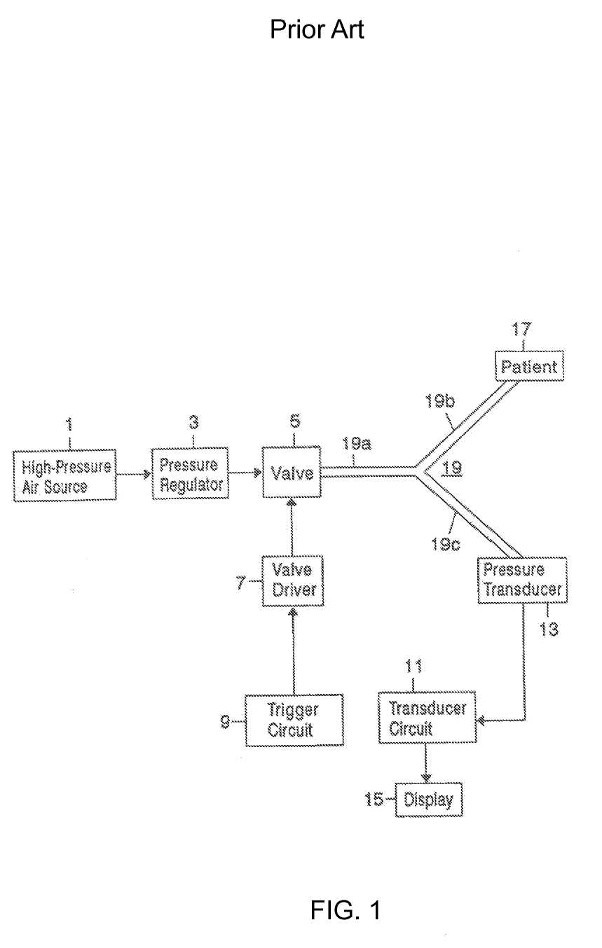

FIG. 1 is a view of a prior art apparatus. This device is as follows.

### PRIOR ART TECHNOLOGY

Device for testing larynx only
All-in-one device
Offered two stimulus modes: continuous and pulsatile
Simple analogue layout, only had to pick mode and output pressure
Offered limited increments in pressure outputs (0.1 mmHg)
Valve was solenoid (no bearings)

### Device Problems

Assumes multi-neuron reflex has no other inputs to influence it
   Resulted in very inconsistent data, as new studies show both the receptor and peripheral nervous system play critical roles.
Pressure output highly unstable
   Results in both inconsistent results and possible safety hazards to patient
Air was directly pulled from outside using compressor
   Result was device could not be used in a sterile environment, special precautions needed to be taken to prevent contamination, and maybe a potential source of bacterial infection.
   Mechanical noise allowed patients to anticipate stimulus resulting in inaccurate results.
   Noise could affect results by increasing patient's stress levels.
Device output often did not match input
Design was not modular
   Could not be easily improved with future versions
Too heavy to easily transport
Analogue user interface with few options.

### Laryngeal Mechanosensor (LMS)

FIG. 2 is a view of the laryngeal mechanosensor stimulator according to this invention. The device has the following features and improvements.

### Key Features

Dual solenoid valves for more control
Excess pressure relieve valve
Sterile delivery of stimulus, ability to be able to be used in a sterile environment
High end microcontroller with feedback control
More accuracy, faster ramp time (time to correct pressure) and more robustness (compatibility with patients)
Mucosensory input
   Computer user interface with basic and advanced control options.
   User interface is hard coded with delimiters to prevent doctor from administering unsafe pressures
   Design is highly modular

### Design Improvements

Designed from physiological standpoint to isolate receptor and nervous system output
   Studies will be novel, very accurate and high impact
Relief valve guarantees high pressures cannot be used with patients
Feedback control ensures both high degree of accuracy and patient safety
   Software includes delimiters to ensure doctor cannot program unsafe outputs
Device uses medical grade air source to provide air pressure
   Closed loop system can be sterilized to eliminate bacterial source
Design is digital and highly modular
   Can patch through fast updates to customers if there are any problems
   Allows for easy implementation of addition signal inputs and outputs
   Opens up entire market for add-ons
Computer interface allows freedom to run test from anywhere in the room
Muscosensory input is novel

Typical laryngeal mechanoreceptor physiology is well known.
Laryngeal/Pharyngeal receptor
   Thick myelinated rapidly conducting Aβ fibers responsible for pressure and vibration
   Thin Aδ and unmyelinated thin fibers responsible for temperature.
Laryngeal Adductor Reflex
   Multi-neuron involuntary reflex
   Stimulation of laryngeal/pharyngeal mucosa (stimulation of internal branch of the superior laryngeal nerve) leads to reflex closure of vocal cords.

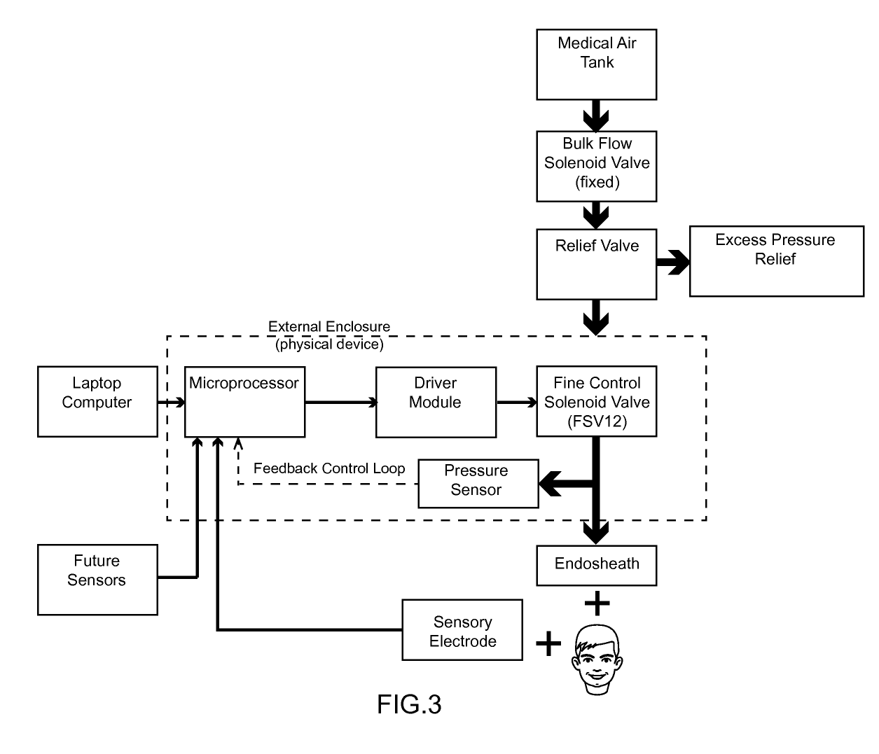

FIG. 3 shows a sensory electrode. The diagram includes both the electrode for measuring superior laryngeal nerve and an input for future sensors.

### EXAMPLE I

Initially, medical grade air, either from a tank or wall source, is fed into the device. Prior to reaching the primary components, the air passes through an excess pressure relief valve. This way the incoming air pressure can be limited physically to protect the patient in case of a malfunction upstream of the device.

Within the enclosure, the air reaches a precisely controlled system in which a two valve feedback controlled system provides delicate control for desired output pressures. The solenoid valves are driven by signals from the driver module and microprocessor, which receive command signals by way of user input through the software interface. The user can program both the pressure magnitude and the duration of each test on a very easy to use Guided User Interface. Thus the user directly controls how much air is allowed to pass through the fine control valves, and finally reach the patient (via laryngoscopy).

To ensure greater safety, a couple of measures were taken. A transistor-transistor logic (TTL) input enables the valve to be closed regardless of the analog input, which allows for greater safety and control of the device as the valve can be safely completely closed when not in use. A pressure sensor located downstream of the solenoid valve provides feedback of the air output back to the software. The Feedback Control System (FCS) creates a circuit loop which communicates back to the microprocessor the results in a steady and accurate pressure output over the duration of the stimulus presentation. Lastly all components shall be sterile.

### EXAMPLE II

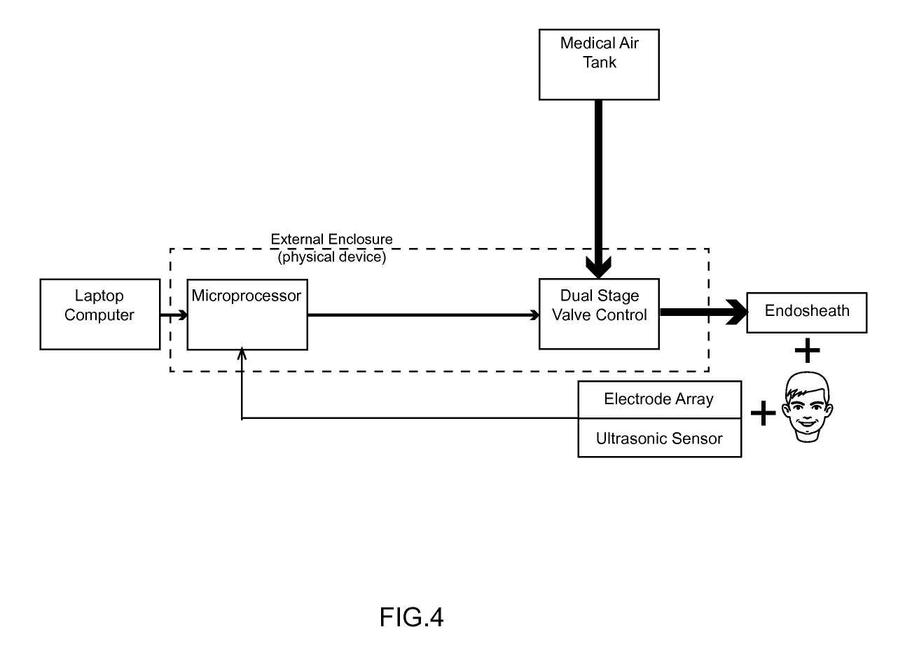

FIG. 4 shows an ultrasonic sensor integrated into the laryngeal mechanoreceptor. FIG. 4 shows an augmented electrode manifold for the purpose of robustly location a peripheral nerve. With the integration of an ultrasonic sensor, it becomes possible to identify the corresponding artery or vein, and therefore locate the nerve itself. The primary use case would be as a supplement to the localization of the nerve by the identification of the corresponding blood vessel. For example, for the internal branch of the superior laryngeal artery is co-located where branches of the superior thyroid artery penetrate the thro-hyoid membrane. Similar pairings of blood vessels and commonly used peripheral nerves for diagnostic purposes, such as the median or sural nerves occur. Thus, this augmented electrode manifold will enable the attending physician to precisely locate the desired nerve for diagnostic purposes, such that its signal can be measured with minimal interference.

The device improves targeting of peripheral nerves such as the internal branch of the superior laryngeal nerve using surface electrodes. Two major short comings of surface electrodes use are electrode placement and interference from the surrounding muscular tissue. Needle electrodes are an alternative, but are limited by a patient acceptance especially with neck placement. It is very difficult to get a clear neural signal with topical electrodes due to the poor signal to noise ratio. Traditional amplification and filtering of the signal will not suffice, as amplification would maintain the signal to noise ratio, and filtering could distort the action potential signal. Optimizing electrode placement will improve the signal to noise ratio without the introduction of distortion.

This issue also generalizes to EMG nerve conduction studies, which often require the usage of invasive insertion electrodes to diagnose nervous system functional status. Given even insertion electrodes require close placement to the nerve for signal acquisition, this sometimes results in multiple insertions for each patient. This can be a painful procedure, such that patients have been known to refuse treatment on this basis alone. Therefore, there is a clear need for a system that can precisely locate the position of commonly used peripheral nerves for electro-diagnostic purposes.

This solution couples an ultrasonic sensor with the EMG electrode manifold. For example, the superior thyroid artery and vein accompany the internal branch of the superior laryngeal nerve as it exits the larynx through a hole in the thyrohyoid membrane, such that physicians have used this artery in the past to locate the nerve. Given the density difference of the surrounding tissues: blood vessels, cartilage, and the surrounding fascia, ultrasonic sensor can be used to resolve the position of superior thyroid artery and vein and thereby the internal branch of the superior laryngeal nerve. Thus, by resolving the co-located blood vessel we are able to more precisely locate the internal branch of the superior laryngeal nerve enhancing the signal to noise ratio—in a very cost effective manner.

Because the nerve runs horizontally in the same plane before turning vertically, the electrode manifold incorporates an array of electrodes to capitalize on this anatomic regularity. The reliability of the directionality of the commonly used peripheral nerves makes electrode array-placement feasible. Once the nerve located, it is very reasonable to incorporate others to not only improve signal to noise robustness but to permit assessment of nerve transmission velocity.

Note this technology generalizes very well to other peripheral nerves, such as the median, sural, and peroneal nerves. Many peripheral nerves used in EMG studies have an artery or vein in close proximity; as such, the same ultrasonic sensing technology could be used to locate the location of the nerve. Therefore, it is believed that this system could also be offered as a standalone nerve targeting system, to be used for peripheral nerve sensory and stimulatory electrodes.

This device integrates an ultrasonic sensor as an analogous input, and then couples the sensor and electrode together into a single manifold.

The electrode array will permit qualitative assessment of nerve conduction velocity through the delays identified as the action potential passes and is detected in each subsequent equidistant placed sensors in the array. To give an indication of location, we would use a harmonic generator. We would take the absolute value of the difference in measured density versus expected blood density, and have the system “beep” at a duty cycle that reflects proximity. It could also be possible to give a visual indicator on the LMS's Guided User Interface, but we would most like leave that within the ‘Advanced’ tab for simplicity.

It is additionally proposed that actuation mechanism be offered for physician who are interested in using insertion electrodes. While the typical insertion electrode is painful due to the geometry, there do exist painless needles, such as those used in acupuncture. Given that all that is necessary for nervous signal acquisition is a quality conducting surface, a similarly thin needle could be integrated directly into the manifold, with corresponding actuator. This actuator would then deliver the painless insertion electrode precisely into the patient at a location of the nerve. In this case of one time use electrodes, the actuator could be a simple spring loaded mechanism. In the use case of repeated use electrodes, the actuator could be a DC motor, where feedback control could be used to determine proper electrode insertion depth. Note that the electrode would have a fine point, followed with greatly widening base, to create a physical barrier to unsafe insertion depths.

The above detailed description of the present invention is given for explanatory purposes. It will be apparent to those skilled in the art that numerous changes and modifications can be made without departing from scope of the invention. Accordingly, the whole of the foregoing description is to be construed in an illustrative and not a limitative sense, the scope of the invention being defined solely by the appended claims.

We claim:

1. An apparatus that provides feedback controlled pressure output that delivers a highly controlled and accurate air stimulus into the larynx for diagnostic use, comprising:
a supply of medical air;
an excess relief valve;
receiving medical air from the supply;
a first pressure regulator receiving medical air from the relief valve exhausting to the outside environment;
a second pressure regulator receiving medical air downstream from first pressure regulator;
a drive module connected to the second pressure regulator configured to deliver a highly controlled and accurate air stimulus into a larynx for experimental and diagnostic use from the second pressure regulator;
a microprocessor connected to the pressure regulators, sensors and driver module configured to control the pressure regulators and driver module; and
a computer and software connected to the microprocessor configured to control the microprocessor and monitor internal pressure sensors.

2. An apparatus according to claim 1 further comprising an endosheath receiving medical air from the second pressure regulator.

3. An apparatus according to claim 1 wherein the first pressure regulator is a bulk flow solenoid valve.

4. An apparatus according to claim 1 wherein the second pressure regulator is a fine control solenoid valve.

5. An apparatus according to claim 1 further comprising a feedback control loop between the second pressure regulator and the microprocessor.

6. An apparatus according to claim 5 wherein the feedback control loop includes a pressure sensor.

7. An apparatus according to claim 1 configured to isolate receptor and nervous system output.

8. An apparatus according to claim 1 wherein the relief valve is configured to prevent high pressure from the upstream medical air source.

9. An apparatus according to claim 1 wherein the software further comprises delimeters to prevent programming unsafe outputs.

10. An apparatus according to claim 1 further comprising a transistor-transistor logic (TTL) input that enables the valve to be closed regardless of the analog input.

11. An apparatus according to claim 5 wherein a pressure sensor located downstream of the solenoid valve provides feedback of the air output back to the software.

12. An apparatus according to claim 5 wherein the feedback control loop creates a circuit loop which communicates back to the microprocessor the results in a steady and accurate pressure output over the duration of a stimulus presentation.

13. An apparatus according to claim 1 configured to determine when it is safe to engage in swallowing following surgery or anesthesia.

14. An apparatus according to claim 1 configured to determine mucosal receptor functions thereby accurately for the first time separating peripheral from central neurosensory lesions of the pharynx (hypopharynx).

15. An apparatus according to claim 1 configured to determine mucosal receptor functions thereby accurately for the first time separating peripheral from central neurosensory lesions of the pharynx (hypopharynx) with greater diagnostic precision the presence and treatment of laryngopharyngeal reflux (LPR) vs. gastroesophageal reflux (GERD).

16. An apparatus according to claim 1 configured to determine mucosal receptor functions thereby accurately for the first time separating peripheral from central neurosensory lesions of the pharynx (hypopharynx) reduction of laryngeal mucosal swelling.

17. An apparatus according to claim 1 configured to determine mucosal receptor functions thereby accurately for the first time separating peripheral from central neurosensory lesions of the pharynx (hypopharynx) extra-glottic from glottis vocal disorders.

18. An apparatus according to claim 1 configured to determine mucosal receptor functions thereby accurately for the first time separating peripheral from central neurosensory lesions of the pharynx (hypopharynx) results of anti-reflux therapy (GERD).

19. An apparatus according to claim 1 further comprising a sensory electrode for measuring laryngeal nerve input.

20. An apparatus according to claim 19 including the sensory electrode and further comprising an input for future sensors.

21. An apparatus that provides feedback controlled pressure output that delivers a highly controlled and accurate air stimulus into the larynx for diagnostic use, comprising:
a supply of medical air;
a dual stage valve control receiving medical air from supply;
a microprocessor connected to the dual stage valve control;
a computer and software connected to the microprocessor configured to control the microprocessor; and
a single manifold of ultrasonic sensor integrated with electrode array wherein the manifold is configured to deliver a highly controlled and accurate air stimulus into a larynx for diagnostic use.

22. An apparatus according to claim 21 further comprising a feedback control loop between the manifold and the microprocessor.

23. An apparatus according to claim 21 wherein the ultrasonic sensor is an analog input.

24. An apparatus according to claim 21 wherein the electrode array is an EMG electrode array.

25. An apparatus according to claim 21 further comprising an endosheath receiving medical air from the dual stage valve control.

26. An apparatus that provides feedback controlled pressure output that delivers a highly controlled and accurate air stimulus into the larynx for diagnostic use, comprising:
a supply of medical air;
a dual stage valve control receiving medical air from supply;
a microprocessor connected to the dual stage valve control;
a computer and software connected to the microprocessor configured to control the microprocessor; and
an ultrasonic sensor wherein the ultrasonic sensor is configured to deliver a highly controlled and accurate stimulus into a larynx for diagnostic use.
